(19) United States
(12) Patent Application Publication
(10) Pub. No.: US 2023/0181378 A1
(43) Pub. Date: Jun. 15, 2023

(54) Infant Diapers with Pockets

(71) Applicant: Jamie Morris, Las Vegas, NV (US)

(72) Inventor: Jamie Morris, Las Vegas, NV (US)

(21) Appl. No.: 17/549,472

(22) Filed: Dec. 13, 2021

(51) Int. Cl. A61F 13/15; A61F 13/84; A61F 13/551

(52) U.S. Cl. CPC A61F 13/15585; A61F 13/84; A61F 13/5515; A61F 2013/55125

(57) ABSTRACT

Infant Diapers With Pockets are carefully crafted to pack all baby essentials, such as wet wipes and disposable diaper bags, in one compact diaper. It has two essential built-in storage pockets. The resealable pocket located in the middle of the diaper has wet wipes, and the pocket found on the lower right region is used to carry disposable diaper bags. These diapers are created with an ultra smooth, lightweight, and baby sensitive material that will ensure the baby is safe and protected in all situations. The disclosure additionally includes a sealed pocket disposed on an outside of the disposable diaper and comprising the disposables bag attached at a point on an outside thereof to an inside of the sealed pocket, wherein an outside of the disposables bag deploys from the pocket to enclose the disposable diaper and an inside of the disposables bag becomes its outside.

### BACKGROUND

A diaper (American and Canadian English) or a nappy (Australian English, British English, and Hiberno-English) is a type of underwear that allows the wearer to urinate or defecate without using a toilet, by absorbing or containing waste products to prevent soiling of outer clothing or the external environment. When diapers become wet or soiled, they require changing, generally by a second person such as a parent or caregiver. Failure to change a diaper on a sufficiently regular basis can result in skin problems around the area covered by the diaper. Diapers are primarily worn by infants, toddlers who are not yet toilet trained, and by children who experience bedwetting. They are also used by adults under certain circumstances or with various conditions, such as incontinence.

### SUMMARY OF THE INVENTION

Infant Diapers With Pockets are carefully crafted to pack all baby essentials, such as wet wipes and disposable diaper bags, in one compact diaper.

Forgetting baby wipes is a common mishap that most parents face on a weekly basis which can lead to frustration and being put in an uncomfortable position for both the baby and the parents. Considering baby care includes various products, it is a probable situation that parents become flustered and overwhelmed when going on visits when they have a limitation of space and nowhere to store all of their baby products.

Ingenious and practical, Infant Diapers With Pockets expands on the initial basis of a regular diaper. Infant Diapers With Pockets have two essential built-in storage pockets. The resealable pocket located in the middle of the diaper has wet wipes, and the resealable pocket found on the lower right region is used to carry disposable diaper bags. Infant Diapers With Pockets is created with an ultra smooth, lightweight, and baby sensitive material that will ensure the baby is safe and protected in all situations. It is a fully equipped diaper that will ease the process of cleanup for parents and will ensure the baby is properly sanitized at all times.

Infant Diapers With Pockets is the only product of its kind as it includes two additional functions that enhance the utility of modem day diapers. Infant Diapers With Pockets are uniquely designed to decrease the chance of parents forgetting any essential baby needs, and act as a product that ensures the hygiene of both the infant and the parent.

The disclosed disposable diaper includes a resealable pocket disposed on an outside of the disposable diaper and configured for dispensing fresh wipes sealed therein and for disposing used wipes resealable therein. The disclosure also includes a disposables bag having an inside and an outside and a reseal able opening configured to arrange in an initial flat and compressed configuration. The disclosure additionally includes a sealed pocket disposed on an outside of the disposable diaper and comprising the disposables bag attached at a point on an outside thereof to an inside of the sealed pocket, wherein an outside of the disposables bag deploys from the pocket to enclose the disposable diaper and an inside of the disposables bag becomes its outside.

### BRIEF DESCRIPTION OF THE DRAWINGS

FIG. 1 is a frontal view of the diaper with pockets in accordance with an embodiment of the present disclosure.

FIG. 2 is a perspective view of a disposables bag deployed from a sealed pocket to enclose a used diaper and wipes in accordance with an embodiment thereof.

FIG. 3 is the cross sectional view AA to AA of FIG. 1 in accordance with an embodiment of the disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

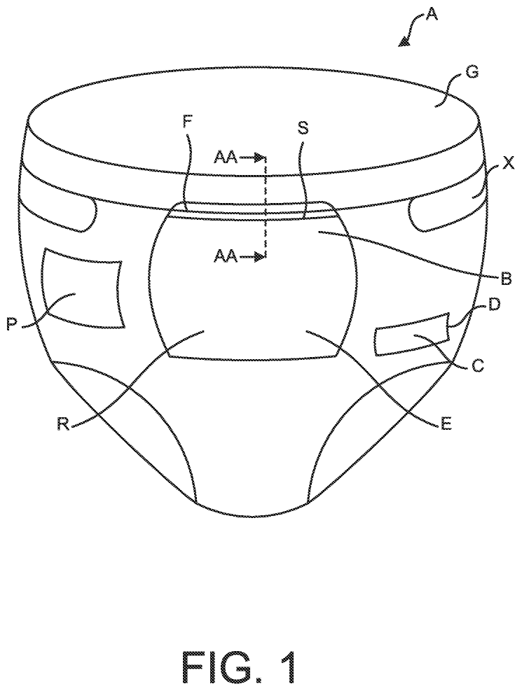

FIG. 1 is a front perspective view of the diaper with pockets in accordance with an embodiment of the present disclosure. The view depicts the disposable diaper A, the resealable pocket B, the sealed pocket C, the seal D which is factory set to avoid tampering by a child or infant, the moist wipes E contained in the resealable pocket B whether fresh or used and a rash cream packet R contained on or in the resealable pocket B according to model types and manufacturing constraints. The view also includes the flap F of the resealable pocket B, the seal 5, the personal item(s) pocket P, the elastic G and the second elastic or supporting band X, and the cross section AA to AA.

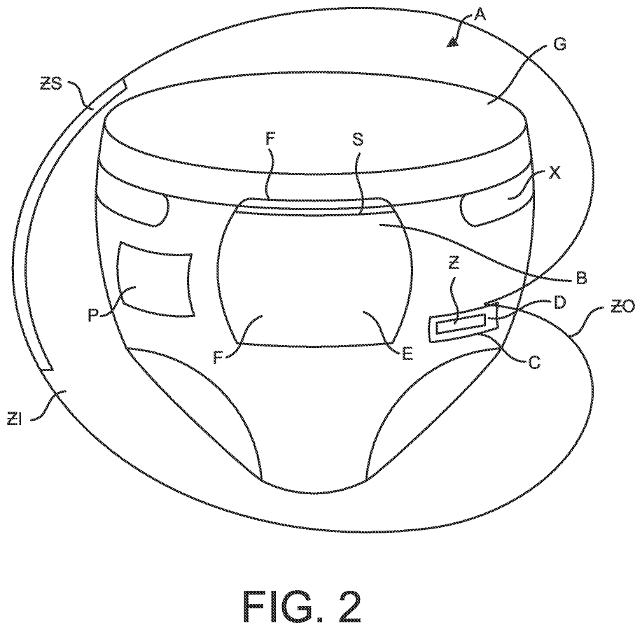

FIG. 2 is a perspective of a disposables bag deployed from a sealed pocket to enclose a used diaper and wipes in accordance with an embodiment thereof. In addition to the same and similar limitations referenced in FIG. 1, the outside of the disposables bag ZO in deployment which predeployment is an inside surface of the disposables bag, the inside surface ZI of the disposables bag in deployment is adjacent the disposable diaper A and the disposables seal ZS are depicted. Therefore a used disposable diaper is enclosed for disposal with less handling and a hygienic manner and method via the disposables bag Z.

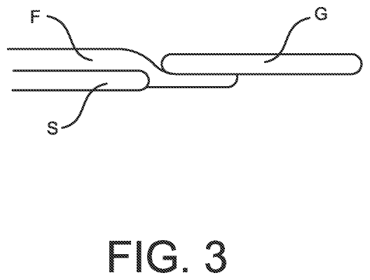

FIG. 3 is the cross sectional view AA to AA of FIG. 1 in accordance with an embodiment of the disclosure. The view shows the flap F tucked into the resealable opening S defined between the elastic G and the open edge of the resealable pocket B. The flap F is tucked underneath the elastic G to form a resealable edge thereof. A similar seal is disclosed for the personal pocket P. The sealed pocket C discourages tampering with the disposables bag predeployment.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A disposable diaper comprising:
a resealable pocket disposed on an outside of the disposable diaper and configured for dispensing fresh wipes sealed therein and for disposing used wipes resealable therein;
a disposables bag having an inside and an outside and a resealable opening configured to arrange in an initial flat and compressed configuration; and
a sealed pocket disposed on an outside of the disposable diaper and comprising the disposables bag attached at a point on an outside thereof to an inside of the sealed pocket, wherein
an outside of the disposables bag deploys from the pocket to enclose the disposable diaper and an inside of the disposables bag becomes its outside.

2. The disposable diaper of claim 1, further comprising a rash cream packet disposed adjacent the resealable pocket.

3. The disposable diaper of claim 1, wherein an opening for the resealable pocket is disposed on a wider side of the resealable pocket in relation to its sides.

4. The disposable diaper of claim 1, wherein the resealable pocket is large enough to store a nominal 4 moist wipes.

5. The disposable diaper of claim 1, wherein the resealable pocket seals in a scent from used wipes.

6. The disposable diaper of claim 1, wherein the resealable pocket has a subpocket for fresh wipes and a subpocket for used wipes.

7. The disposable diaper of claim 1, wherein the resealable pocket is configured to seal in a moisture in the fresh wipes and in the used wipes.

8. The disposable diaper of claim 1, wherein the sealed pocket is disposed on an outside of the resealable pocket.

9. The disposable diaper of claim 1, wherein the resealable pocket is disposed on an outside of the sealed pocket.

10. The disposable diaper of claim 1, further comprising a rash cream packet disposed on an outside of the resealable pocket.

11. The disposable diaper of claim 1, further comprising a rash cream packet disposed on an outside of the sealed pocket.

12. The disposable diaper of claim 1, wherein the sealed pocket is detachable from the disposable diaper.

13. The disposable diaper of claim 1, wherein the resealable pocket is detachable from the disposable diaper.

14. The disposable diaper of claim 1, further comprising a rash cream packet which is detachable and reattachable from an outside of the disposable diaper.

15. The disposable diaper of claim 1, further comprising a personal item(s) pocket disposed thereon and having an opening adjacent an elastic waistband thereof.

16. The disposable diaper of claim 1, wherein one of the resealable and the sealed pockets are disposed on a back side of the disposable diaper.

17. The disposable diaper of claim 1, wherein the resealable and the sealed pockets are disposed on a backside of the disposable diaper.

18. The disposable diaper of claim 1, wherein on of the resealable and the sealed pockets are disposed on a front side of the disposable diaper.

19. The disposable diaper of claim 1, wherein the resealable and the sealed pockets are disposed on a front side of the disposable diaper.

20. The disposable diaper of claim 1, wherein an opening of the resealable pocket is disposed adjacent an elastic waistband of the disposable diaper as a seal therefore.
